(19) United States
(12) Patent Application Publication
(10) Pub. No.: US 2006/0190013 A1
(43) Pub. Date: Aug. 24, 2006

(54) Curved closure jaw arrangement for a medical clip applier device

(75) Inventor: Pavel Menn, Marblehead, MA (US)

Correspondence Address: GREENBLUM & BERNSTEIN, P.L.C., 1950 ROLAND CLARKE PLACE, RESTON, VA, 20191, US

(73) Assignee: MICROLINE, INC., Beverly, MA (US)

(21) Appl. No.: 11/061,606

(22) Filed: Feb. 22, 2005

(51) Int. Cl. A61B 17/10

(52) U.S. Cl. 606/142

(57) ABSTRACT

An elongated jaw assembly for pinching closed a medical clip in an elongated medical clip-applying device. The assembly has an elongated bifurcated set of jaw members on a set of jaw arms. The assembly has a first end and a second end with a curvilinear rail arranged on an edge of each of the arms. A movable cinch member is slidably disposed on the jaw assembly to effect both closure and spreading apart of said jaw members on a tissue pinching medical clip supported therebetween.

### BACKGROUND OF THE INVENTION

1. Field of the Invention

This invention relates to medical clip devices and more particularly to pinching jaw arrangements for those devices which are utilized to pinch a tissue portion. The disclosures of U.S. patent application Ser. No. 11/000,177 filed on Nov. 30, 2004, entitled “Medical Clip Applier Arrangement”, which claims the benefit of U.S. patent application Ser. No. 10/085,737, filed on Feb. 28, 2002, which matured into U.S. Pat. No. 6,840,945, both of which are commonly assigned with the present application, are expressly incorporated herein by reference in their entireties.

2. Description of Background Information

Medical clip devices are utilized to pinch a body tissue. The clip device usually comprises an elongated support arm having a distal end comprising a pair of squeezable jaws. Such medical clip devices may be seen in U.S. Pat. No. 6,277,131 to KALIKOW, and U.S. Pat. No. 6,306,141 to MEADE, the disclosures of each of which are expressly incorporated herein by reference in their entireties.

### SUMMARY OF THE INVENTION

It is a feature of the present invention to provide a pinching jaw arrangement for a medical clip applier device in which the jaws and slider portion which effects the pinching thereof, be manufactured in a simpler and more cost-effective manner.

It is a further feature of the present invention to provide a squeezing jaw arrangement for a medical applier device which permits a larger mechanical advantage by a larger force on the end of the closing clip which the required force performing a smaller eye thereon is substantially higher.

It is a further feature of the present invention to provide a squeezable jaw arrangement which does not have interruptions in its track for guidance, to minimize manufacturing cost.

It is a further feature of the present invention to provide better control and guidance of those squeezable jaws which jaws are also stronger and stiffer to reduce the possibility of those jaws being bent during a procedure.

It is a yet further feature of the present invention to provide a jaw arrangement which will give improved occlusion closure with respect to eye gaps and leg gaps.

One feature of the present invention includes an elongated, bifurcated split jaw assembly which includes an elongated first arm and an elongated second arm having a distalmost end for squeezing a medical clip therebetween. The jaw assembly includes a proximal end where the arms are joined at a connector tab which would be utilized for attachment to an elongated beam as shown in U.S. Pat. No. 6,840,945. The first and second arms are manufactured in an opposed relationship and have a cinch member slidably disposed thereon. Each jaw has an inner side with a track thereon for slidably receiving a medical clip therewithin. Such a medical clip is preferably of generally U-shaped and is effected at the distal end of those jaw pairs by a pusher arrangement not shown in this present application. Each jaw member has an arm portion thereon, respectively. The upstanding rail is elongated, and forms a stretched out or elongated S-shape, which rail extends rearwardly to the proximal end of the jaw assembly.

The cinch member is advanceable distally and returnable proximally by a movable connector member, not shown in the present application but identified and described in U.S. Pat. No. 6,840,945. The cinch, is an elongated member having an upper portion of curvilinear shape and a lower jaw engaging planar portion with a pair of jaw member receiving tracks therein. The receiving tracks include a curvilinear cam surface on the exterior sides thereof on its distal end.

A curvilinear spreader block, such as for example, an ellipse, or preferably of circular shape, is disposed on the distalmost edge of the planar surface and has a gap between its respective adjacent cam surfaces. That gap defines the gap which slidingly receives the upstanding rail portion of each first and second jaw arm.

Distal and proximal motion of the cinch relative to the jaw assembly effects a biasing of the jaws toward one another or away from one another. Distal advance of the cinch member effects biasing of the jaw members against one another to squeeze a clip therebetween. Proximal movement of the cinch member effects with respect to the jaw assembly effects the release of the jaws and hence the spreading apart of the jaws of the jaw assembly so as to enable those jaws to pushably receive a further medical clip sequentially fed therebetween, as recited in the patents incorporated herein by reference.

The use of an upstanding rail on the outer periphery of each particular jaw arm at the most transverse external portion thereof maximizes the pinching capabilities at the distal end of each jaw, so as to squeeze and close a medical clip therebetween. The long smooth curved surface of the cam on each track, without any interruptions thereon, permits a smooth and seamless squeezing force to those distal jaws, so as to effectively maximize the effectiveness of any medical clip squeezed thereby.

The elongated, extended and drawn out “S” shape curved nature of the rail on each jaw member is thus effective to provide a strong “moment arm” on the legs of a clip supported in the track grooves on the face of each respective jaw member, to press and bend those clip legs in a more complete and pinched manner.

A feature of the present invention thus includes an elongated jaw assembly for pinching closed a clip in an elongated clip-applying device. The assembly comprises an elongated bifurcated set of jaw members on a set of jaw arms. The assembly has a first end and a second end. A curvilinear rail is arranged on an edge of each of the arms. A movable cinch member is slidably disposed on the jaw assembly to effect closure and spreading apart of the jaw members on a clip supported therebetween. The curvilinear rail is arranged in an upstanding manner on each of the arms. The curvilinear rail is formed into an elongated generally “S” shape on each of the arms. The cinch has a curved upper surface and a generally planar jaw-engaging lower surface.

The planar jaw-engaging lower surface has a pair of rail-receiving tracks on a first end thereof. The cinch member is arranged to slide on the rail-receiving tracks. Each of the pair of rail-receiving tracks has an outer edge including an outer cam surface. Each of the pair of rail-receiving tracks has an inner edge including a spreader block. The spreader block is preferably of curved shape, for example, of a generally circular configuration on the planar surface. The spreader block is disposed between the outer cam surfaces. Movement of the cinch member toward the first end effects pinching of a clip disposed between the jaw members. Movement of the cinch member toward the second end effects unbiasing and subsequent spreading apart of the jaw members.

Another feature of the present invention includes a method for pinching closed a clip in a substantially elongated clip-applying device having a first end and a second end. The method includes receiving the clip supported between a substantially elongated bifurcated set of jaw members on a set of jaw arms, in which each jaw member has a curvilinear rail positioned on each jaw arm.

The cinch is advanced toward the first end to result in a squeezing movement of each of the curvilinear rails, thus effecting biasing of the jaw members and resulting in the clip being pinching between the jaw members. The cinch is advanced in a second direction to effect the release of the jaw members. A second clip may then be received between the elongated bifurcated set of jaw members on the set of jaw arms. During advancement in the first and second directions, the cinch slides on a pair of rail-receiving tracks. The release of the jaw members causes the jaw members to open. The curvilinear rail is vertically disposed on each of the arms and is formed into a substantially elongated “S” shape on each of the arms.

Another feature of the present invention includes an elongated jaw assembly for pinching closed a clip, in an elongated clip-applying device, in which the assembly has a first end and a second end. A substantially elongated pair of jaw members is provided on a set of jaw arms. A curvilinear rail, formed into a substantially elongated “S” shape, is arranged on an edge of each of the arms in a vertically disposed manner. A movable cinch member is slidably disposed on the jaw assembly to effect closure and opening of the jaw members. Movement of the cinch member toward the first end effects pinching of the medical clip disposed between the jaws members and movement of the cinch member toward the second end effects a release and opening of the jaw members.

Other exemplary embodiments and advantages of the present invention may be ascertained by reviewing the present disclosure and the accompanying drawings.

### BRIEF DESCRIPTION OF THE DRAWINGS

The present invention is further described in the detailed description which follows, in reference to the noted plurality of drawings by way of non-limiting examples of certain embodiments of the present invention, in which like numerals represent like elements through the several views of the drawings, and wherein:

FIG. 1 is a perspective view of a jaw assembly with a cinch member shown thereon, according to an aspect of the present invention;

FIG. 2 is a top plan view of the jaw assembly and cinch therewith, shown in FIG. 1, according to an aspect of the present invention;

FIG. 3 is a top plan view of the jaw assembly, without the cinch member shown thereon, according to an aspect of the present invention;

FIG. 4 is a perspective view of the bottom side of the jaw assembly with the cinch member shown thereon, according to an aspect of the present invention; and

FIG. 5 is a perspective view of the bottom side of a cinch member, according to an aspect of the present invention.

### DETAILED DESCRIPTION OF THE PREFERRED EMBODIMENTS

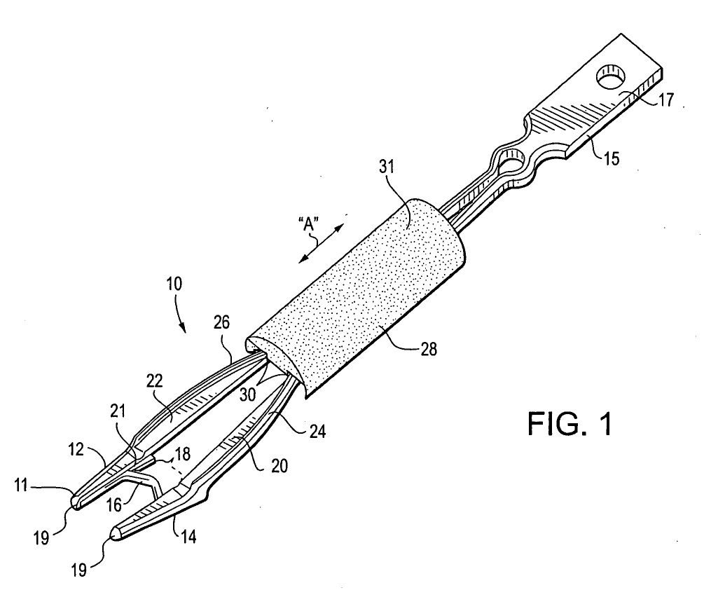

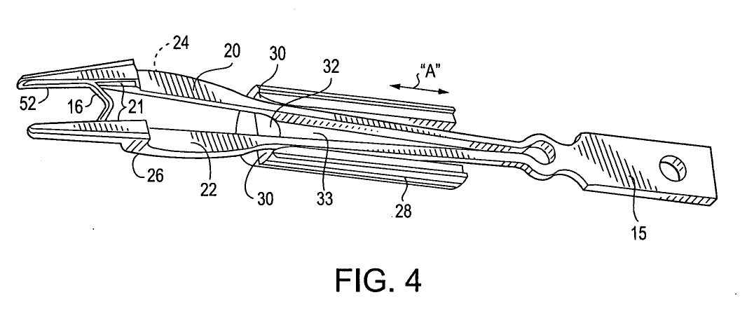

Referring now to the drawings in detail, and particularly to FIG. 1, there is shown the present invention which comprises an elongated, bifurcated split jaw assembly 10 which includes an elongated first arm 12 and an elongated second arm 14 each having a distalmost end 11 for squeezing a medical clip 16 therebetween. The jaw assembly 10 includes a proximal end 15 where the arms 12 and 14 are joined at a connector tab 17 which would be utilized for attachment to an elongated beam as shown in U.S. Pat. No. 6,840,945. The first and second arms 12 and 14 are manufactured in an opposed relationship and have a cinch member 28 slideably disposed thereon. Each arm 12 and 14 has an inner side or face 18 with a track or groove 21 thereon for slidably receiving a medical clip 16 therewithin, as represented in FIGS. 1 and 4.

Such a medical clip 16 is preferably of generally U-shape and is effected at the distal end 19 of those jaw pairs 12 and 14 by a pusher arrangement, not shown for clarity, in this present application. Each jaw member 12 and 14 has an arm 20 and 22 portion respectively, thereon.

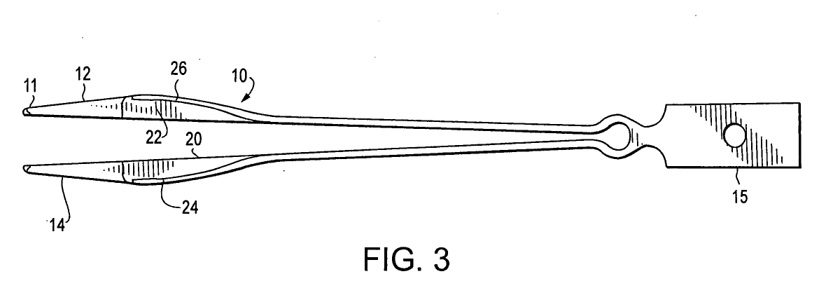

An elongated, stretched out “S” shaped upstanding rail 26 and 24 is arranged on the outer peripheral edge of each arm 22 and 20, respectively, as shown in FIGS. 1, 2 and 3, extends rearwardly towards the proximal end 15 of the jaw assembly 10.

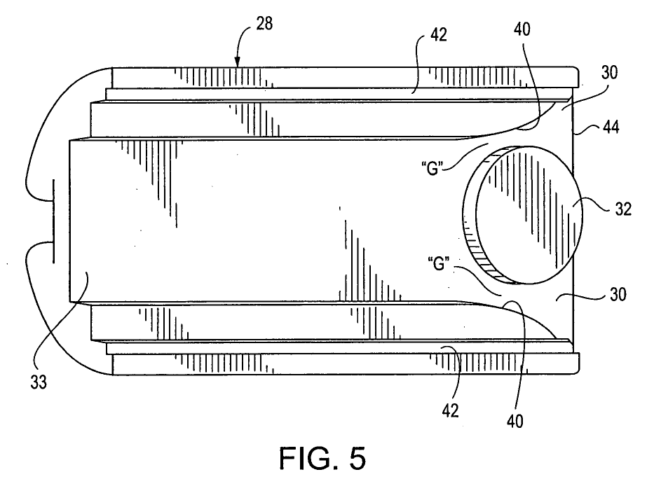

The cinch member 28 is movable distally and movably returnable proximally by a movable connector member, not shown in the present application, but such connector member being identified and described in the incorporated U.S. Pat. No. 6,840,945. The cinch member 28, is an elongated member having an upper portion 31 of curvilinear shape and a lower jaw-engaging planar portion 33 (FIGS. 4 and 5) with a pair of jaw-member receiving-tracks 30 therein, as represented in FIGS. 1 and 5. The receiving tracks 30 include a curvilinear cam surface 40 on each of the exterior sides 42 thereof adjacent its distal end 44 (FIG. 5).

A smoothly curved spreader block 32, for example, of elliptical or generally circular shape, as represented in FIG. 5, is disposed on the distalmost edge of the planar surface 33 and has a gap “G” defining the track 30, between the block 32 and its respective adjacent cam surfaces 40. That gap “G” defines the tracks 30 which slidingly receive the upstanding rail portions 26 and 24 of each first and second jaw arms 22 and 20.

Distal and proximal motion of the cinch member 28 relative to the jaw assembly 10, as indicated by arrow “A” effects a biasing of the jaws 12 and 14 toward one another or away from one another. Distal advance of the cinch member 28 effects biasing of the jaw members 12 and 14 against one another to squeeze a clip 16 therebetween. Proximal movement of the cinch member 28 effects with respect to the jaw assembly 10 effects the release of the jaws 12 and 14 and hence the spreading apart of the jaws 12 and 14 of the jaw assembly 10 so as to enable those jaws 12 and 14 to pushably receive a further medical clip 16 sequentially fed therebetween, as recited in the aforementioned patents incorporated herein by reference.

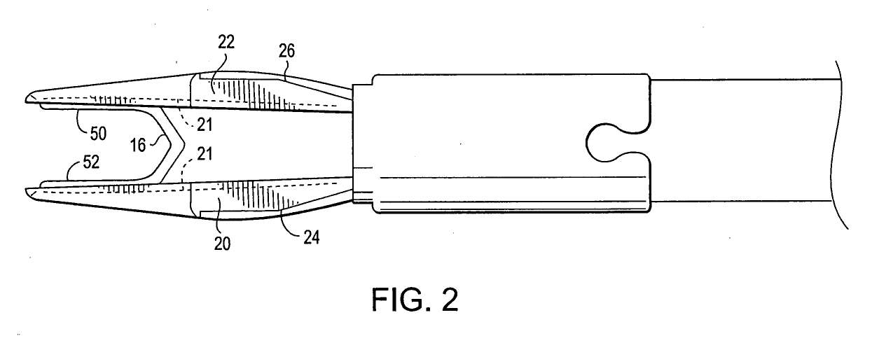

The use of a curvilinear, “S” shaped upstanding rail 26 and 24 on the outer periphery of each particular jaw arm 22 and 20 at the most transverse external portion thereof maximizes the pinching capabilities at the distal end 11 of each jaw 12 and 14, so as to squeeze and close a medical clip 16 therebetween. Each rail 26 and 24 is arranged in an upstanding manner (e.g., vertically) on a respective jaw arm. In other words, when viewing FIG. 2, the rails 26 and 24 generally extend out of the page. The long smooth curved surface 40 of the cam defining a side of each track 30, without any interruptions thereon, permits a smooth and seamless squeezing force applied to those distal jaws, so as to effectively maximize the effectiveness of the pinch on any medical clip 16 squeezed thereby.

The elongated, extended and drawn out “S” shape curved nature of the rails 26 and 24 on each jaw member arm 22 and 20 respectively, is thus effective to provide a strong “moment arm” on the legs 50 and 52 of a clip 16 supported in the track grooves 21 on the face of each respective jaw member 12 and 14, to press and bend those clip legs 50 and 52 in a more complete and pinched manner.

Although the invention has been described with reference to an exemplary embodiment, it is understood that the words that have been used are words of description and illustration, rather than words of limitation. Changes may be made, within the purview of the appended claims, as presently stated and as amended, without departing from the scope and spirit of the present invention in its aspects. Although the invention has been described herein with reference to particular means, materials and embodiments, the invention is not intended to be limited to the particulars disclosed herein. Instead, the invention extends to all functionally equivalent structures, methods and uses, such as are within the scope of the appended claims.

What is claimed is:

1. An elongated jaw assembly for pinching closed a clip, in an elongated clip-applying device, the assembly having a first end and a second end, comprising:
an elongated bifurcated set of jaw members on a set of jaw arms;
a curvilinear rail arranged on an edge of each of said arms; and
a movable cinch member slidably disposed on said jaw assembly to effect closure and spreading apart of said jaw members on a clip supported between said jaw members.

2. The jaw assembly as recited in claim 1, wherein said curvilinear rail is positioned on an upper surface of each of said arms.

3. The jaw assembly as recited in claim 2, wherein said curvilinear rail is formed into an elongated generally “S” shape on each of said arms.

4. The jaw assembly as recited in claim 1, wherein said cinch has a curved upper surface and a generally planar jaw-engaging lower surface.

5. The jaw assembly as recited in claim 4, wherein said planar jaw-engaging lower surface has a pair of rail-receiving tracks on an end thereof.

6. The jaw assembly as recited in claim 4, wherein said cinch member is arranged to slide on said rail-receiving tracks.

7. The jaw assembly as recited in claim 5, wherein each of said pair of rail-receiving tracks has an outer edge comprised of an outer cam surface.

8. The jaw assembly as recited in claim 5, wherein each of said pair of rail-receiving tracks has an inner edge comprised of a spreader block.

9. The jaw assembly as recited in claim 8, wherein said spreader block is of a generally circular configuration on said planar surface.

10. The jaw assembly as recited in claim 7, wherein said spreader block is disposed on said planar surface, between said outer cam surfaces.

11. The jaw assembly as recited in claim 1, wherein movement of said cinch member toward the first end effects pinching of the clip disposed between said jaws members.

12. The jaw assembly as recited in claim 11, wherein movement of said cinch member toward the second end effects unbiased spreading apart of said jaw members.

13. A method of pinching closed a clip in a substantially elongated clip-applying device, the device having a first end and a second end, the method comprising:
receiving the clip between a substantially elongated bifurcated set of jaw members on a set of jaw arms, each jaw member having a curvilinear rail positioned on each jaw arm;
advancing a cinch toward the first end to perform a squeezing movement of each of the curvilinear rails, and effect biasing of the jaw members such that the clip is pinched between the jaw members; and
advancing the cinch toward the second end to release the jaw members.

14. The method as recited in claim 13, further comprising receiving a second clip between the elongated bifurcated set of jaw members on the set of jaw arms.

15. The method recited in claim 13, wherein the cinch slides on rail-receiving tracks during advancement toward the first and second ends.

16. The method recited in claim 13, wherein the release of the jaw members opens the jaw members.

17. The method recited in claim 13, wherein the curvilinear rail is vertically disposed on each of the jaw arms.

18. The method recited in claim 13, wherein the curvilinear rail is a substantially elongated “S” shape on each of the arms.

19. An elongated jaw assembly in an elongated clip-applying device, said assembly having a first end and a second end, comprising:
a substantially elongated pair of jaw members on a set of jaw arms;
a curvilinear rail arranged on an edge of each of said arms; and
a movable cinch member slidably disposed on said jaw assembly to effect closure and opening of said jaw members.

20. The jaw assembly as recited in claim 19, wherein said curvilinear rail is vertically disposed on each of said arms.

21. The jaw assembly as recited in claim 19, wherein said curvilinear rail is a substantially elongated “S” shape on each of said arms.

22. The jaw assembly as recited in claim 19, wherein movement of said cinch member toward the first end effects pinching of a clip disposed between said jaws members.

23. The jaw assembly as recited in claim 19, wherein movement of said cinch member toward the second end effects a release of said jaw members.
